(19) United States
(12) Patent Application Publication
(10) Pub. No.: US 2012/0215126 A1
(43) Pub. Date: Aug. 23, 2012

(54) DETERMINING DYNAMIC AIRWAY RESPONSE IN A SUBJECT

(76) Inventors: Noam Gavriely, Haifa (IL); Peter Camilleri, Springvale (AU)

(21) Appl. No.: 13/389,662

(22) PCT Filed: Aug. 6, 2010
(86) PCT No.: PCT/AU2010/000995

§ 371 Date: May 9, 2012

(30) Foreign Application Priority Data
Aug. 12, 2009 (AU) 2009903786

(51) Int. Cl. A61B 7/00; A61B 5/091

(52) U.S. Cl. 600/538; 600/586

(57) ABSTRACT

A method for assessing a broncho-dynamic response in a subject includes introducing a sound signal having known characteristics into the airway of a subject, detecting one or more responsive sound signals at one or more locations on the thorax, administering a broncho-effector to the subject, and determining the subject's response to the broncho-effector by monitoring the one or more responsive sound signals before and after administering the broncho-effector. Changes in the responsive sound signal characteristics indicate the subject's broncho-dynamic response to the broncho-effector. Apparatus for assessing broncho-dynamic response in a subject is also provided.

### FIELD OF THE INVENTION

The present invention relates to methods, systems and apparatus for determining a dynamic airway response in a subject. In particular, it relates to use of sound signals to monitor a subject's response to e.g. chemical, environmental and physical broncho-effective factors.

### BACKGROUND TO THE INVENTION

Asthma is a disorder affecting the airways of the lungs. Over 2 million Australians have asthma, and the disorder is one of the most common reasons for hospitalization of children in Australia. People with asthma have highly sensitive airways that narrow in response to certain “triggers” and this leads to breathing difficulty and reduced airflow in and out of the lungs. Narrowing of the airway is caused by inflammation and swelling of the airway lining, tightening of the airway muscles, production of excess mucus or a combination of these. At present the cause of asthma is not known and although there are treatments available, there is no cure.

Asthma is a widespread and chronic health problem. The causes of asthma are not understood although sufferers often have a family history of asthma, eczema or hay fever. Other risk factors include exposure to smoke in early childhood and for unborn babies, mothers who smoke during pregnancy. Asthma can begin at any age and severity of attacks can change over time.

Asthma symptoms can include a dry, irritating, persistent cough, particularly at night, early morning, with exercise or activity or in colder temperatures; chest tightness; shortness of breath and wheezes emanating from the airways. Triggers are believed to include colds and flu, exposure to cigarette smoke, exercise/activity, inhaled allergens (e.g. pollens, moulds and dust mites), environmental contaminants (e.g. dust, pollution and smoke), changes in temperature and weather, certain medications (e.g. aspirin), chemicals and strong smells (e.g. perfumes, cleaners), emotional factors (e.g. laughter, stress) and some foods and food preservatives, flavourings and colourings (uncommon).

Asthma treatments include medications which are generally classed as (i) preventers; (ii) relievers; and (iii) symptom controllers. Preventers reduce airway inflammation, make the airways less sensitive to trigger factors, reduce the redness and swelling inside the airways and dry up the mucous. Preventers are prescribed to reduce the risk of asthma attacks. Preventer medications formulated for inhalation and/or as oral medications often include corticosteroids, e.g. budesonide and methyl prednisolone and leukotriene receptor antagonists e.g. Montelukast. The effect of preventers is cumulative. Therefore, it can take some weeks before there is a noticeable improvement in symptoms.

Relievers or rescue medications provide short term relief from asthma symptoms within minutes e.g. salbutamol, terbutaline. Acting as a broncho-dilator, they relax the muscles around the airways for up to four hours allowing air to move easily through the airways and are typically used during an asthma “attack”. Symptom controllers (also referred to as long acting relievers) e.g. salmeterol and formoterol help to relax the muscles around the airways for up to 12 hours. They are usually taken on a daily basis and typically prescribed for asthma sufferers who take regular inhaled ‘steroid’ preventers. The reversibility of the airway narrowing by inhaled short-acting medications is an important characteristic of asthma and is often used as a diagnostic indicator.

Accurate asthma diagnosis, including determining the severity of attacks can be difficult because subjects are often asymptomatic at the time of examination. Diagnostic tests are available such as spirometry and forced oscillatory techniques (FOT).

Spirometry testing, also known as a “pulmonary function test” or PFT, is an objective test used to measure the volume and maximal flows of inspired and expired air from the lungs. The results are plotted on a graph and indicate the degree of airway narrowing. The reliability of results obtained using spirometry is dependent on the ability of the subject to cooperate and follow instructions and is not therefore suitable for infants or young children, the elderly or individuals suffering from certain types of neuromuscular and mental illnesses. In addition, peak flow readings obtained using spirometry do not always indicate the true state of the small airways, and are therefore of limited value in diagnosis.

Forced oscillatory technique (FOT) involves inflating and deflating the lung by volume cycling at the mouth or chest surface. Using FOT, low frequency oscillatory pressures and the simultaneously generated flows are measured in a rigid tube held in the subject's mouth. The complex ratio (i.e. real and imaginary components) between the two measured parameters is calculated as function of the oscillatory frequency. This is used to determine the input impedance of the lung and has been a popular method for testing lung capacity in pre-school children due to its non-invasiveness. Using this technique, flow is determined indirectly using pressure measurements. However, these measurements can be unreliable because the tube can influence the propagation characteristics of the pressure wave, the oscillations of the oral and pharyngeal soft tissues, and the need to hold a mouthpiece.

In many cases, the methods of measuring the responses of the airways require cooperation of the subject who is unable to perform the test and are so unreliable that physicians may prescribe asthma medication without prior definitive diagnosis with the intent to monitor the subject over a period of time. If the subject is responsive to the treatment, the physician deduces that the subject is an asthma sufferer. This “empirical” approach to diagnosis of a severe and chronic condition is unsatisfactory, as are the methods used to monitor a subject's response to treatment regimes. Therefore, there is a need for effective assessment of asthmatics, treatment methods and ongoing monitoring of their effectiveness.

The discussion of the background to the invention included herein including reference to documents, acts, materials, devices, articles and the like is intended to explain the context of the present invention. This is not to be taken as an admission or a suggestion that any of the material referred to was published, known or part of the common general knowledge in Australia as at the priority date of any of the claims.

### SUMMARY OF THE INVENTION

Viewed from one aspect, the present invention provides a method for assessing a broncho-dynamic response in a subject, including the steps of: introducing a sound signal having known characteristics into the airway of a subject; detecting one or more responsive sound signals at one or more locations on the thorax; administering a broncho-effector to the subject; and determining the subject's response to the broncho-effector by monitoring the one or more responsive sound signals before and after administering the broncho-effector; wherein changes in the responsive sound signal characteristics indicate the subject's broncho-dynamic response to the broncho-effector.

The sound may be introduced into the airway intermittently or continuously in any suitable manner such as via the mouth, one or both nares, endotracheally or using a face mask to name a few. Monitoring the responsive sound signals may also occur during administration of the broncho-effector. Preferably, a reference signal is obtained from a transducer on the subject's neck, just below the glottis. Using the method, the acoustic transmission of the subject can be determined where e.g. a given decrease in acoustic transmission at a particular frequency or frequency band in response to the broncho-effector is used to quantify a broncho-constrictive response in the subject and a given increase in acoustic transmission at a particular frequency or frequency band in response to the broncho-effector is used to quantify a broncho-dilating response in the subject. Preferably, the subject's response to the broncho-effector is intermittently or continuously monitored according to the method, over a period of time.

In a preferred embodiment, the response is corrected to compensate for effects on the responsive sound signal which are attributable to changes in lung volume due to respiration. This may include one or both of: characterising the changing volume-effect of respiration on responsive sound signal magnitude and applying a magnitude correction factor to the responsive sound signal based on the magnitude characterisation; and characterising the changing volume-effect of respiration on responsive sound signal propagation delay and applying a propagation delay correction factor to the responsive sound signal, based on the propagation delay characterisation.

The correction may further involve estimating lung volume at regular intervals and, depending on the lung volume estimate at each interval, applying a correction factor to at least one of the magnitude and the delay of the acoustic transmission determined. The correction factor may be determined based on one or more relationships characterising the changing lung volume effect of respiration on acoustic transmission delay and/or magnitude. The correction factor may vary depending on the sound signal frequencies or bands of frequencies utilised. In a further preferred embodiment, the correcting step is adaptive such that the correction factor varies according to changes in the subject's measured broncho-dynamic response. Such correction may be applied in real time or after monitoring the subject, during post-analysis.

Viewed from another aspect, the present invention provides apparatus for assessing broncho-dynamic response in a subject, the apparatus including: (a) an acoustic signal generator generating a sound signal having known characteristics; (b) a sound introducer for introduction of sound into the subject's airway; (c) one or more sound transducers for detecting a responsive sound signal at one or more locations on the subject's body; (d) a dosimeter for delivering controlled dosages of broncho-effector to the subject; and (e) a processor configured to receive responsive sound signals from the one or more sound transducers and indicate the subject's response to the broncho-effector, based on changes in the responsive sound signal characteristics.

Preferably, the processor is configured to receive the responsive sound signals and indicate the subject's response to the broncho-effector in real-time. Preferably the processor is further configured to calculate acoustic transmission representing the subject's respiratory response to the broncho-effector, and wherein a given decrease in acoustic transmission is used to quantify a broncho-constrictive response in the subject's airway and a given increase in acoustic transmission is used to quantify a broncho-dilating response in the subject's airway. Ideally, the processor is adapted to obtain dynamic measurements of the subject's response, wherein the subject's response is monitored over a period of time.

The dosimeter exposes the subject to one or more broncho-dynamic effectors such as chemical, environmental, and biological effectors to name a few. In a preferred embodiment, the dosimeter determines the controlled dosage based, at least in part, on the subject's response to a previously administered dosage.

In a preferred embodiment, the processor is adapted to compensate the monitored response for the effect on the one or more responsive sound signals of changes in lung volume due to respiration. This may be achieved, for example, by estimating lung volume at regular intervals during monitoring of the responsive signals and applying a correction factor to the calculated acoustic transmission. The correction factor may be based on a relationship characterising the changing volume effect of respiration on the respiratory acoustic transmission. The correction factor may differ, depending on the sound signal frequencies or bands of frequencies being utilised. Furthermore, the correction factor applied to the acoustic transmission may be adaptive, in that it varies according to changes in the subject's measured broncho-dynamic response (e.g. sustained improvement due to treatment).

The processor may also be configured to calculate a value indicating the subject's sensitivity to the administered broncho-effector.

In one embodiment the apparatus further includes a muffler applicable which may be used with one or both of the acoustic signal generator and the sound introducing means to reduce sound emitted to the environment external to the apparatus and the subject. The apparatus may also include one or more of a mask, a nasal cannula in communication with one or both nares, an endotracheal tube and a mouthpiece for introducing the sound signal into the subject's airway.

### BRIEF DESCRIPTION OF THE DRAWINGS

The present invention will now be described in greater detail with reference to the accompanying drawings. It is to be understood that the description of the specific embodiments which follows is not to be taken as limiting on the scope of the invention as defined in the claims appended hereto and does not supersede the generality of the preceding description of the invention.

FIG. 1 is a flow diagram showing steps in a method of determining broncho-dynamic response in a subject.

FIG. 2 is a schematic drawing illustrating components of a system for determining a broncho-dynamic response of a subject according to an embodiment of an invention.

FIG. 3a is a block diagram representing the relationship between input and output signals to a system; FIG. 3b is a graph representing the transfer function of the respiratory system of a subject; FIG. 3c is a graph representing the coherence function of the system represented by FIG. 3b.

FIG. 4 is a graph representing the relationship between a change in transfer function score and change in FEV₁ score.

FIG. 5 is a graph representing the volume dependence of airway patency for a subject's respiratory system.

FIG. 6 is a schematic drawing illustrating components of a system for determining broncho-provocation response of a subject according to another embodiment of the invention.

FIG. 7 is a flow diagram showing steps of a method according to another embodiment of the invention.

FIG. 8 is a schematic sectional drawing of a muffler which may be used with a sound introducing means of the invention.

FIG. 9a illustrates graphically the relationship between propagation delay and change in lung volume. FIG. 9b is a graph representing the dependence of acoustic transmission on changes in lung volume.

FIG. 10 is a graph representing the change in acoustic transmission following the administering of broncho-effectors, Seritide™ and Ventolin™.

FIG. 11 is a graph representing results from another study involving treatment with Salbutamol and Ipratropium Bromide.

FIG. 12 is a graph showing data for a subject who was monitored while undergoing bronchial provocation and bronchodilator reversibility testing as part of their clinical care.

### DETAILED DESCRIPTION

PFT and FOT methods are used to determine if the airway narrowing of a subject is reversible or not. These tests involve a baseline measurement followed by inhalation of a short-acting broncho-dilator and then followed by a measurement of the airway conductivity by the same methods. This test is often called “pre/post broncho-dilator test”. Other tests, such as a bronchial challenge test, may include induction of a mild and well-controlled airway constriction by means that are known to cause such narrowing in general or in the particular subject. Examples include exercise challenges, cold/dry air challenges, allergen challenges or chemical bronchial challenges by use of aerosolized chemicals such as methacholine, histamine, adenosine, manitol and hypertonic saline.

Existing approaches to determining a subject's response to a broncho-effector, e.g. broncho-dilator or broncho-constrictor drugs or a physical perturbation, involve making a static (i.e. once-off) measurement of a subject's lung function before and after the administration of each dose of the drug or other perturbation factor. This is typically done by spirometry using a peak flow meter, or FOT. In most of these procedures the agent used to dilate or provoke the airways is given a sequence of steps. After each step a measurement of PFT or FOT is performed to determine if the subject has already responded, if so then by how much, and if it is safe to administer the next dose. This stepwise process is inherently long and requires intensive and costly labour from the technician.

In contrast, embodiments of the present invention facilitate automated, dynamic and objective assessment of a subject's airway response to a broncho-effector whereby the subject is monitored using acoustic transmission techniques for a continuous period commencing before exposing the subject to the broncho-effector, during the exposure and for a period after exposure.

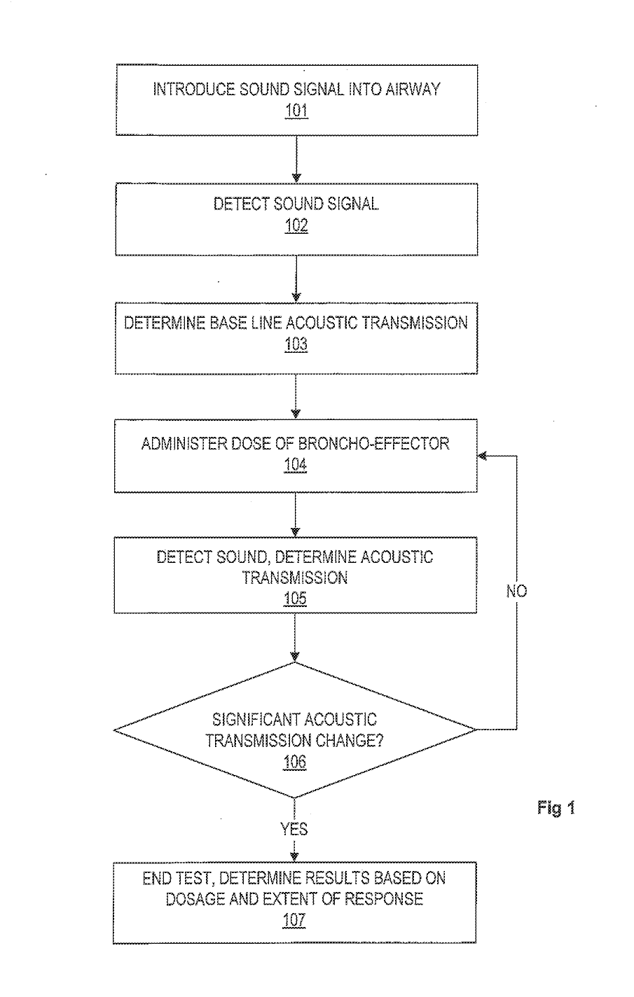

FIG. 1 shows steps involved in a method according to an embodiment of the present invention for determining a subject's broncho-dynamic response to a broncho-effector. This involves introducing a sound signal having known characteristics into the airway of a subject in a step 101. The signal propagates through the lung and chest tissues to the chest wall and is detected in steps 102, 105 using one or more transducers attached to the subject at one or more locations on the thoracic surface. Preferably, baseline acoustic transmission is determined in a step 103. In a step 106, changes in the detected signal are monitored during and after administration of a broncho-effector (step 104). These changes are indicative the subject's airway response (i.e. changes in airway patency due do constriction, dilation or mucus production). The test is terminated in step 107.

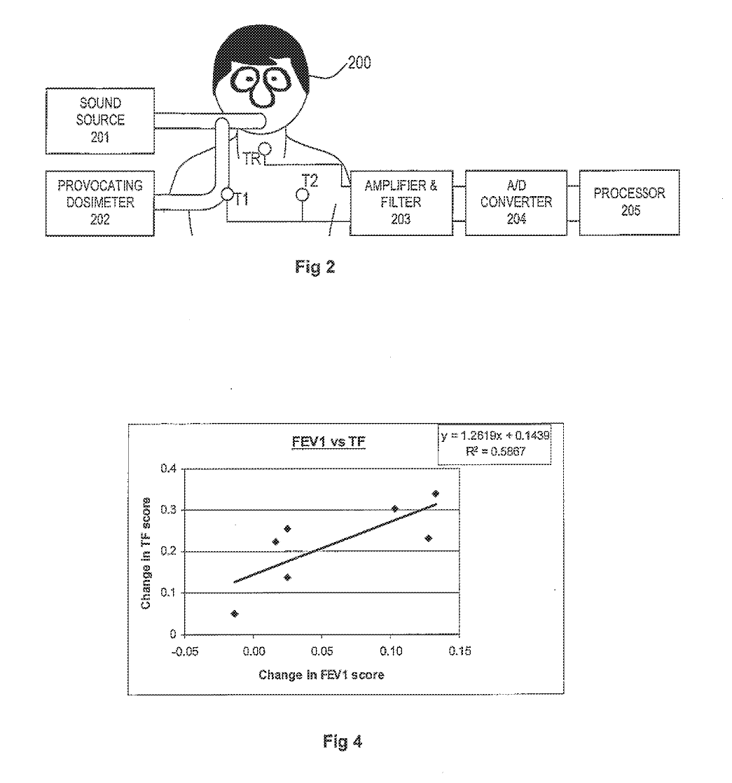

FIG. 2 shows components of a system for determining a broncho-dynamic response of a subject 200 according to an embodiment of the invention. A sound source 201 in the form of e.g. a electro-mechanical device such as a loudspeaker or the like enclosed in an acoustically shielded chamber, connects to sound introducer which interfaces to the subject (e.g. a mouthpiece (tube), nasal tube, mask or endotracheal tube) and introduces a sound signal, having known acoustic characteristics into the subject's airway.

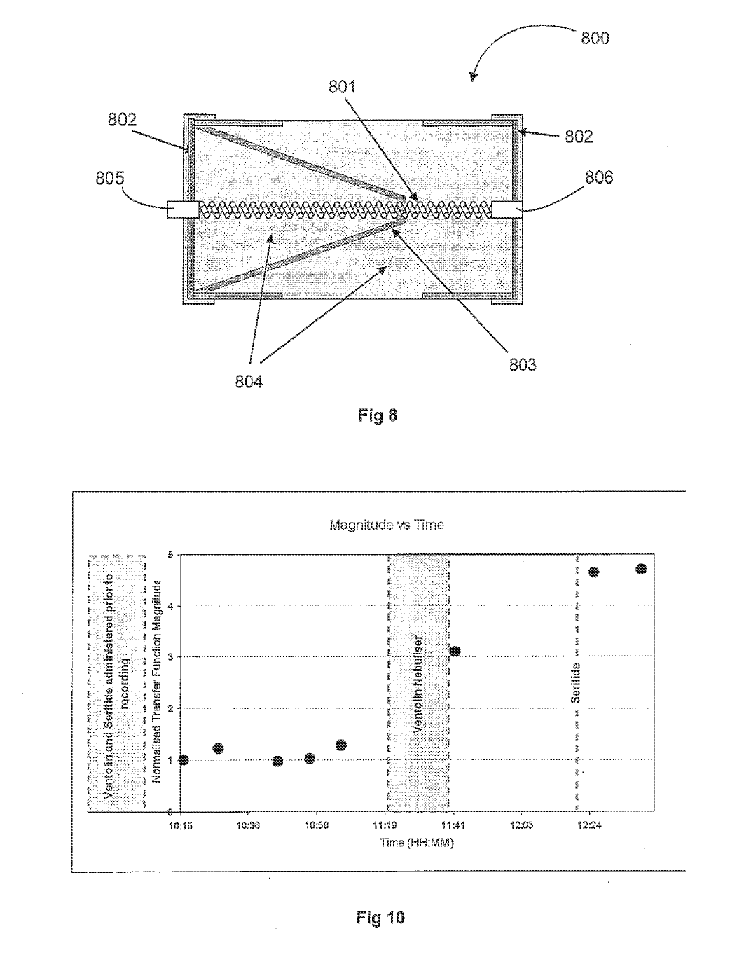

In one preferred embodiment when a mouth piece is used, the sound introducer allows the subject 200 to breathe through the device and incorporates a muffler 800 (FIG. 8) to minimise sound radiated to the surrounding environment. The muffler includes spiral tubing 801 which maintains a passage for the flow of air enabling a patient to breathe whilst enabling sound to radiate outwards into sound absorbing foam 804 which absorbs a large proportion of the sound. A plastic cone 803 retains the spiral tubing in place and reflects incident sound towards the inlet 805 thus minimising sound re-entering the spiral tubing and radiating out to the environment via outlet 806. Dense matting 802 inside the end caps and around the muffler walls provide additional sound absorption. In another preferred embodiment the subject interface also has the provision for oxygen or air delivery or delivery of an oxygen-enriched gas mixture into the subject's airway.

In a preferred embodiment the introduced sound signal is a Maximal Length Sequence (MLS) and more preferably a band limited MLS sequence (e.g. 70 Hz-2000 Hz). It is noted however that any signal such as white noise, multiple sine waves, clicks or swept sine may be used to determine the acoustic transmission of the subject's respiratory system and hence changes in airway patency.

The subject can be monitored at set intervals, e.g. following a dose of a broncho-effector or as in a preferred embodiment, the subject undergoes continuous monitoring. Thus, collected sound recordings from transducers T1 and T2 can be analysed discretely by processor 205 providing updates on the subject's condition at regular intervals (e.g. 1-10 seconds) over a period of time. The processor may be configured to monitor a succession of groups of signals which groupings correspond to administration of one of a series of regular dosages of e.g. a broncho-effector. The regular dosages may be administered e.g. every 1 to 10 seconds during the period of assessment, although it is contemplated that other dosage rates may be utilised.

It will be noted from FIGS. 2 and 6 that processor 205 also receives a sound signal from reference transducer TR which is attached to the subject's neck just below the glottis and over the trachea. The reference transducer provides a reference sound signal which is used by processor 205 to calculate the acoustic transmission (e.g. transfer function) of the subject's chest and respiratory system. By positioning the reference transducer here, the transfer function calculated by processor 205 represents the acoustic transmission of subject's respiratory system including the lower airways, alveolar sacs, thoracic tissues etc., but excludes the effects of the apparatus used to introduce the sound (e.g. loudspeaker, tubing) and the upper airways. Thus, with the reference transducer positioned on the neck just below the glottis the acoustic transmission relates only to the subject's respiratory system between the reference transducer TR and transducers T1 and T2.

The introduced sound signal is transmitted through the subject's airways, lung parenchyma and chest wall tissues such as muscle, ribs, skin etc. and in a step 102, is detected at one or more locations on the thoracic surface. The complex phase-preserving ratio between the detected responsive signal(s) and the TR reference signal (i.e. the complex transfer function) can be used to give a baseline reading of characteristic acoustic transmission (step 103 of FIG. 1) which is indicative of the subject's pulmonary function, i.e. breathing ability before the broncho-effector is administered. During the baseline determination, which may last several minutes (e.g. 2-20 minutes), multiple acoustic transmission evaluations (e.g. every 1-10 seconds) are determined and the average +/−Standard Deviation of these readings is calculated. This may be referred to as the Baseline Average Acoustic Transmission and the Baseline Acoustic Transmission Variability (BATV), respectively.

Once baseline characteristic acoustic transmission data has been obtained, the subject is administered a controlled dosage of a broncho-effector in a step 103. The broncho-effector may be a chemical effector such as broncho-constrictor medication or broncho-dilator medication and this may be introduced into the airway of a subject, e.g. by aerosolising the medication into a mouthpiece or to the surroundings of a subject. Alternatively/additionally, the broncho-effector may include or consist of an environmental effector such as a change in temperature, humidity, particulate matter in the air (e.g. dust or smoke) or an occupational irritant to which the subject is exposed and which can be controlled during assessment of the subject's broncho-dynamic response. Alternatively/additionally the broncho-effector may include a physical/mechanical effector such as exercise, change in posture or eating, administered to the subject by his/her participation. Biological effectors such as allergens, pollen and infective agents may also be utilised in or as the broncho-effector.

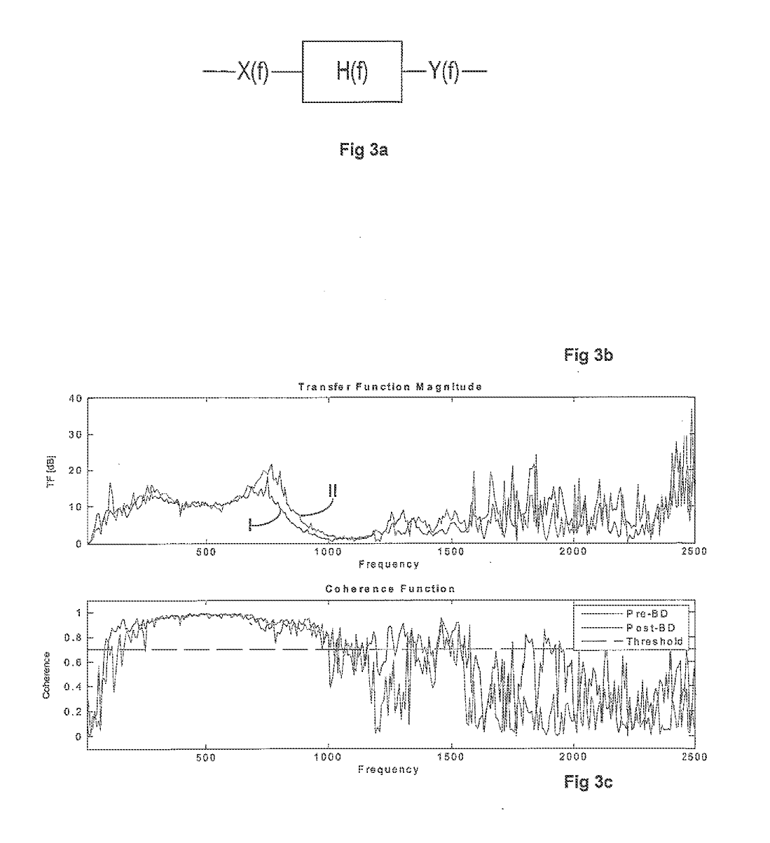

At step 105 detection of the responsive sound signal at the one or more locations is repeated, to determine changes in characteristic acoustic transmission (compared with the baseline readings) which are brought about as a result of the administered dosage of broncho-effector introduced in step 104 by dosimeter 202. The subject's response is monitored using signals from single or multiple and transducers T1, T2 etc., attached to the thorax (e.g. anterior chest or ribs, and/or posteriorly), relative to a reference signal obtained from the TR transducer (attached to the subject's anterior neck or Manubrium Sterni in infants). Sound signals propagating from TR or the sound source and then detected by T1 and/or T2 are referred to as responsive signals, since a comparison of signals taken before, during and after administration of a broncho-effector will indicate the subject's airway response to the effector. One suitable position for a signal transducer T1 or T2 which provides particularly good coherence is on the right side of the chest at the second intercostal space (see FIG. 3c).

The dosimeter 202 may be manually controlled e.g. by a healthcare professional actuating the dosimeter to release a measure of e.g. broncho-dilating chemical which is aerosolised into the subject's airway or the environment or by e.g. increasing the level of exercise. Alternatively, the dosimeter may be controlled by a computerised system which automatically administers a measured dosage of broncho-effector to the subject. In this embodiment, it is preferred that there is a feedback loop between processor 205 and dosimeter 202 so that the level of broncho-dynamic response shown by the subject can be used directly to modify the next dosage administered by the dosimeter as shown in FIG. 6. The next dosage may be calculated by processor 205 and communicated to dosimeter 202, or it may be calculated using a microprocessor device located within the dosimeter itself, which uses sound signal data from processor 205 to determine the dose. A delay mechanism can be used if the effect of the broncho-effector is known to be non-instantaneous.

In a step 106 the acoustic transmission determinations from step 105 are compared with previous readings and e.g. the baseline reading from step 102 and the change in characteristic acoustic transmission corresponding to the applied dose of broncho-effector calculated. If minimal or no response to the applied dose is detected (whereas significant change may be calculated as a function of the BATV e.g. >2 BATV), steps 104 and 105 can be repeated: a further dosage of broncho-effector is administered, and the responsive signal is again detected. If, at step 106, a significant change in acoustic transmission is detected (e.g. a change indicative of <20% FEV₁), or a pre-determined maximum number of e.g. a broncho-provocation agent dosages has been reached, a dose of a reliever may be administered (to counteract the effect of the administered broncho-provocation agent). Further, the subject may be continually monitored until a return to baseline level is achieved.

The assessment is terminated at step 107 after which the results may be evaluated by a healthcare professional. Changes in the responsive sound signal characteristics during monitoring are indicative of the subject's response to the broncho-effectors.

The subject's broncho-dynamic response and more particularly, airway patency, can be determined by passing the responsive signals through an amplifier-filter 203 and Analog-to-Digital (A/D) converter 204 making them suitable for input to digital signal processor 205. In one embodiment of the invention a band pass filter with a high pass component e.g. over 70 Hz is utilised to remove subject movement artefact and a low pass anti-alias filter component e.g. below 4000 Hz is utilised to minimise distortion artefact resulting from sampling of higher frequency components by the A/D converter. The A/D converter may use a sampling rate suitable for evaluating acoustic transmission within the frequency ranges of interest e.g. below 4000 Hz. The inventors have found that suitable sampling rate of e.g. 8000 samples/second satisfies these criteria.

The processor 205 may further filter the sound recordings prior to performing analysis of the signals. In one embodiment of the invention a 70 Hz to 2000 Hz band pass filter is used. The acoustic transmission data of several discrete time sequences may also be averaged to provide time averaged result although many techniques known generally in the art are suitable.

Analysis may also include averaging the acoustic transmission in discrete frequency bands to provide an evaluation for particular frequency ranges. In the previously described embodiment, this may involve averaging the acoustic transmission for each of the frequency ranges e.g. 100 Hz to 500 Hz, 500 Hz to 1000 Hz, 1000 Hz to 1500 Hz and 1500 Hz to 2000 Hz, however any single band or combination of bands may also be used to evaluate acoustic transmission.

FIG. 3a is a simplified block diagram representing the acoustic transmission of the subject where: input signal X(f) corresponds to the signal obtained by reference transducer TR; and output signal Y(f) corresponds to the signal detected by responsive transducer T1 or T2, after the sound signal has travelled a particular pathway to the chest wall. The acoustic transmission function, which represents the system effect on the input X(f) which results in the output Y(f) is represented by “black box” H(f) and is determined by processor 205.

FIG. 3b is a graphical representation of acoustic transmission function indicated by a transfer function magnitude calculated by processor 205 during tidal breathing for a subject's respiratory system pre-and post- administration of a broncho-dilator drug, Salbutamol. The acoustic signal introduced to the subject's airway included frequency components in the range 70 Hz to 2,000 Hz and the responsive signal was monitored using a transducer applied to the subject's chest to determine the acoustic transmission characteristics between the subject's trachea (just below the glottis) and the chest wall. Changes in these characteristics represent the subject's broncho-dynamic response to the broncho-dilator. The traces represented in FIG. 3b represent (I) the transfer function calculated prior to administration of the broncho-dilator and (II) the transfer function calculated immediately after administration of the broncho-dilator.

The validity of the transfer functions in FIG. 3b was verified by determining the coherence of the transfer functions. The respective coherence of the subject's transfer functions is represented in FIG. 3c, as a function of frequency. Here, it can be seen that for this subject coherence is high (i.e. above 0.7) in the frequency band −150 Hz to −1,000 Hz. In the corresponding frequency band in FIG. 3b, it can also be seen that the transfer function magnitude post-administration of broncho-dilator (trace II) is relatively higher than pre-broncho-dilator administration (trace I).

This corresponds with comparison results obtained by traditional spirometry indicating that the change in magnitude of the transfer function corresponds to a change in respiratory function attributable in FIGS. 3b and 3c to increased airway patency. Thus, the present invention has utility for objectively assessing a subject's airway/respiratory response to a broncho-effector. While data is only presented for a single subject in FIGS. 3b to 3c, the inventive technique has been tested on a group of different subjects and the results consistently show that effective administration of broncho-dilator medication is represented by an increase in transfer function magnitude. This result correlated well to a recorded change in subjects FEV₁ score (derived from PFT—see FIG. 4). The degree of increase varied from subject to subject indicating that some subjects responded to the broncho-dilator medication better than others.

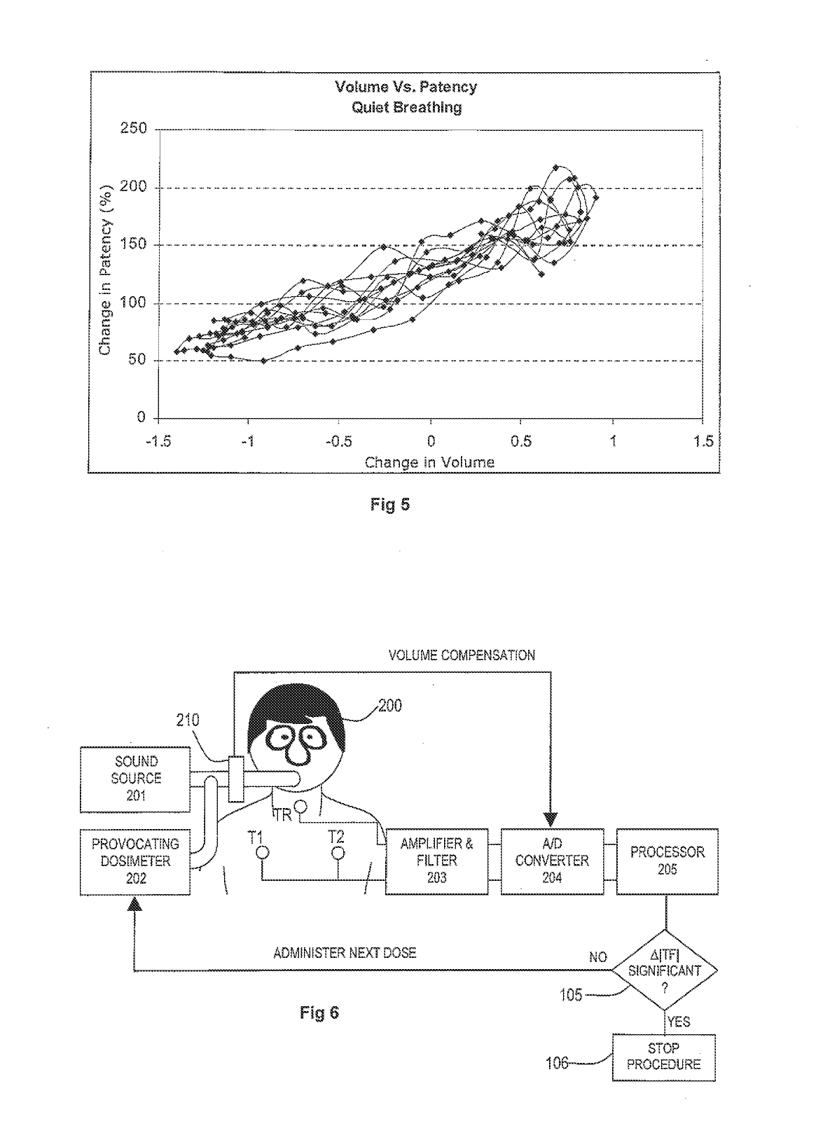

It is to be noted that the data represented in FIGS. 3b to 3c were obtained during an assessment in which the subject was performing quiet, cyclic breathing. However, the inventors have observed that changes to airway patency can also be affected by changes in lung volume which occur during respiration and have discovered that these changes can be characterised. FIG. 5 is a graph representing the relationship between lung volume and “patency” demonstrating that when a subject is in an expiratory phase of breathing (volume decreasing), patency decreases whereas patency increases during the inspiratory phase (where the lung is inflating and the volume is increasing). The inventors attribute this to widening of the lung's airways during inhalation and decreased cross-sectional area during exhalation, in correlation with the change in the lung's volume.

Thus, an embodiment of the invention involves application of a volume correction method to adjust the evaluation of acoustic transmission thereby counteracting the effect of changing lung volume which occurs during respiration. Using this correction method, measured changes in airway patency are attributable solely to the effect of the broncho-effector and are not influenced by changing lung volume. In one embodiment of the correction method, a relationship between acoustic transmission (i.e. airway patency) and changes in lung volume due to respiration is used by processor 205 to adjust the acoustic transmission. This correction may be applied after signal acquisition during post-assessment analysis or to the recorded sound signals as they are acquired. In a preferred embodiment, the correction is applied directly to the acoustic transmission calculation in real time. In order to apply this correction method, the changes in lung volume should be monitored. This may be done e.g. by integrating the flow entering and leaving the lung or by using inductive plethysmography. A modified version of the apparatus of FIG. 2 and which is suitable for applying the volume correction method is shown in FIG. 6.

FIG. 6 additionally shows flowmeter 210 (or other device e.g. pressure sensor) which provides input to processor 205 (via A/D converter 204) indicating where the subject is in the breathing cycle (e.g. inspiration or expiration). Knowing where the subject is in the breathing cycle may be used by the processor to estimate the subject's likely lung volume. However, in a preferred embodiment, flowmeter 210 is used to monitor lung volumes during collection of sound recordings. Baseline measurements of lung volume and acoustic transmission can be used by the processor to develop a calibration formula, look up table or other model or relationship which represents the influence on acoustic transmission which is attributable to lung inflation (and deflation) during respiration.

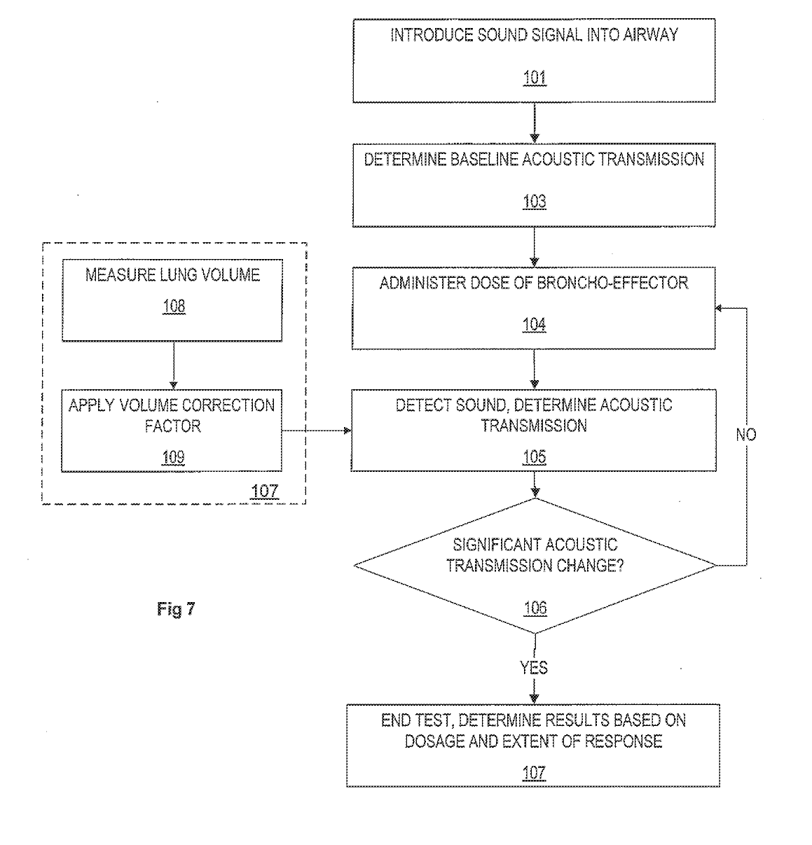

The acoustic transmission determined after administration of the broncho-effector and during the remainder of the assessment can then be corrected according to values in the look-up table or calibration formula for the relevant stage in the breathing cycle. Thus, the corrected acoustic transmission gives a realistic indication of the subject's broncho-dynamic response to the broncho-effector which is not tainted by the changing lung-volume effect of respiration. FIG. 7 also shows steps 105 and 106 from FIG. 1. Further, the flow diagram of FIG. 7 shows additional steps (grouped at 107) involved with compensating for changes in lung volume including: measuring lung volume (at 108) and applying a volume correction factor (at 109).

The baseline data used to model the effect on acoustic transmission of changing lung volume during respiration may vary according to the frequency ranges being investigated whereby different correction factors may be used to correct the acoustic transmission data at different frequencies.

Interestingly, in addition to observing the changing lung-volume effect on acoustic transmission (and hence transfer function magnitude) brought about by respiration, the inventors have discovered that the effect of respiratory volume changes can alter during the course of assessment for a particular subject. This may be attributable to changes in mean lung volume and/or changes in maximum and/or minimum lung volume during the test. These physiological changes may be brought about during the period of assessment e.g. as relief is provided by administration of a broncho-dilating effector, or as the airway is constricted in response to administration of a broncho-provocation agent/effector.

For example, during an assessment where the broncho-effector being administered is a broncho-dilator, the subject's lung capacity may improve as the airway patency improves, thereby increasing the maximum tidal volume compared to pre-assessment (and pre-administration of the broncho-dilator) where the lung was in a relatively contracted state. Further, the administration of the broncho-dilator may also have an anti-mucosal effect in the lung further improving the subject's respiratory performance.

Accordingly, in some embodiments, the correction method applied by the processor is “adaptive” and capable of being modified, during assessment according to changes in the subject's physiology. Thus, a correction “factor” applied to the acoustic transmission according to the correction method may be characterised in many different ways e.g. linear, quasi-linear, exponential, logarithmic, or represented by some other mathematical relationship. The correction factor may be a function of frequency, as well as a function of lung volume.

In addition, the correction method may be performed by reference to a look-up table containing data which represents the effect on acoustic transmission of lung volume during various stages of respiration (e.g. inspiration vs. expiration). The data modelling the effect of lung volume changes during respiration may be representative of a particular category of subject (e.g. based on age, gender, size, lung condition). Alternatively, the data may be specific to the subject undergoing assessment.

Data used for making a correction which is specific to the subject being assessed may be obtained during collection of baseline readings of acoustic transmission (FIG. 1; step 102). That is, the processor 205 uses the baseline readings of acoustic transmission and respiratory volume for a number of breathing cycles (e.g. 3 cycles) as indicative of the effect on the responsive signal attributable to volume changes during respiration (since the data is obtained pre-administration of broncho-effector).

In addition to the changing volume-effect of lung inflation and deflation on acoustic transmission magnitude, the inventors have discovered a volume-effect which influences the propagation delay of acoustic transmission. The propagation delay variations are believed to be attributable to changes in sound signal propagation velocity which are brought about by, in the case of administration of a broncho-dilator, an increase in airway patency.

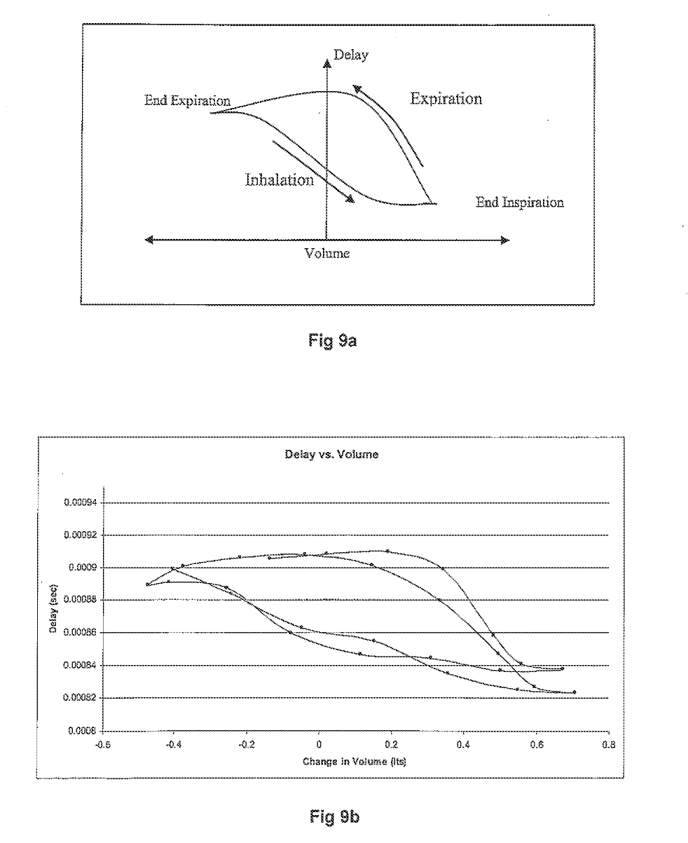

FIG. 9a illustrates the relationship between propagation delay and change in lung volume. The delay is calculated e.g. by a measure of the location of the peak of the impulse response. The hysteresis curves of FIGS. 9a and 9b suggest that inhalation and exhalation possess different transmission mode characteristics. Interestingly minimum lung volume demonstrates a larger delay indicating that the trend present between lung volume and delay is not merely a product of distance due to chest wall expansion (if this were the case the large volume would be representative of a larger delay) but rather that the airways and parenchyma are playing a significant role.

It has been proposed that peripheral airway resistance may be the main constituent behind this result. Many of the smaller airways, like bronchioles, lack the structural support of cartilaginous tissue and derive their structural support from surrounding lung parenchyma; as such the smaller components of the airway lumen are readily distensible and collapsible. As lung inflation occurs, radial traction increases and transmural pressure becomes more negative; resultantly peripheral airway diameter increases, reducing frictional resistance to airflow. During this phase of the breathing cycle it has been observed that a decrease in sound propagation delay (increase in sound velocity) occurs.

Conversely, an increase in sound propagation delay (decrease in velocity) was observed during expiration. During this phase, radial traction decreases, transmural pressure become less negative and airway diameter decreases all of which result in an increase in frictional resistance to airflow. This leads the inventors to a hypothesis that delay time is directly related to airway resistance and would account for the observed trends between delay times and lung volume, with the hysteresis of the delay loop demonstrating the reactive nature of the airways. The graph shown in FIG. 9b demonstrates this process over two breath cycles.

FIG. 10 is a graphical representation of acoustic transmission indicated by a transfer function magnitude calculated by processor 205 during tidal breathing for a subject's respiratory system who presented at an emergency department with shortness of breath and exacerbation of Asthma, The graph demonstrates an increase in acoustic transmission following clinical care incorporating the administration of a bronchodilator, Seritide™, Ventolin™ and oxygen.

FIG. 11 shows results from another study where a subject with a diagnosis of exacerbation of asthma was monitored using the inventive technique during treatment and a subsequent recovery phase. As shown in FIG. 11, a gradual improvement in airway patency is observed following treatment with Salbutamol and Ipratropium Bromide.

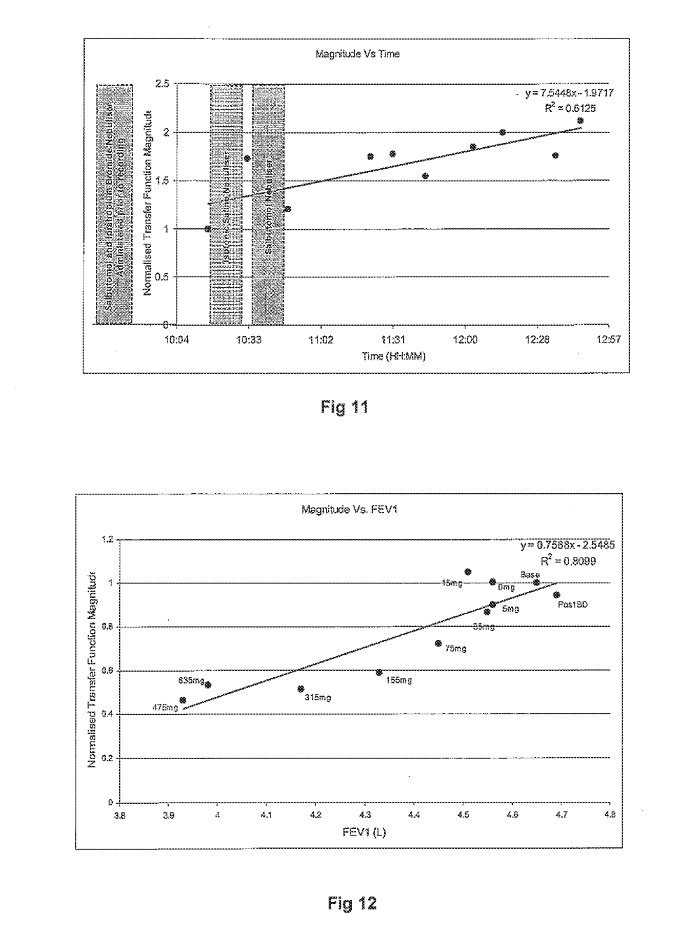

FIG. 12 shows data for a subject who was monitored while undergoing bronchial provocation and bronchodilator reversibility testing. This subject recorded a 13% reduction in FEV₁ as a result of broncho-provocation. This has been correlated against measurements of patency using the inventive technique as illustrated in FIG. 12 which shows a statistically significant correlation between a change in patency (evidenced by change in transfer function magnitude) and a change of FEV₁. Thus, reduction in patency as determined using the inventive technique corresponds to reductions in FEV₁; conversely, an increase in patency following the administration of the broncho-dilator (BD) corresponds to an increase in FEV₁. This is a positive indicator for use of the inventive technology for objectively monitoring a subject's broncho-dynamic response to a broncho-effector.

In addition to monitoring the subject's acoustic transmission, the measurements can be combined with other observations including observations relating to sounds emanating from within the subject, in response to exposure to the broncho-effector. Such sounds may include coughs, wheezes, crackles, rhonchi and the like. Methods and apparatus for monitoring these indigenous sounds are described in U.S. Pat. Nos. 6,168,568 and 6,261,238 the contents of which are hereby incorporated herein by reference.

The present invention is not susceptible to or at least improves upon the shortcomings of spirometry as described in the Background, since the subject's response is monitored objectively and continuously based on the acoustic transmission characteristics of the subject's respiratory system and full subject cooperation is not required.

It is to be understood that various other modifications, additions and/or alterations may be made to the parts previously described without departing from the spirit of the present invention as outlined herein and in the claims appended hereto.

1. A method for assessing a broncho-dynamic response in a subject, including the steps of:
introducing a sound signal having known characteristics into an airway of the subject;
detecting one or more responsive sound signals at one or more locations positioned on a thorax of the subject;
administering a broncho-effector to the subject; and
determining a response to the broncho-effector by monitoring the one or more responsive sound signals from the subject at a first time before administering the broncho- effector and a second time after administering the broncho-effector; wherein changes in the one or more responsive sound signals detected at the first time and the one or more responsive sound signals detected at the second time indicate the broncho-dynamic response to the broncho-effector.

2. A method according to claim 1 further including the step of obtaining a reference signal from a transducer positioned and brought into contact with a neck of the subject, just below a glottis.

3. A method according to claim 1 including the step of determining a baseline measurement of acoustic transmission of the subject prior to administering the broncho-effector.

4. A method according to claim 1 further including monitoring the one or more responsive sound signals during the step of administering the broncho-effector.

5. A method according to claim 1 including the step of determining an acoustic transmission of the subject, and further wherein:
(i) a given decrease in acoustic transmission in response to the broncho-effector is used to quantify a broncho-constrictive response in the subject; and
(ii) a given increase in acoustic transmission in response to the broncho-effector is used to quantify a broncho-dilating response in the subject.

6. A method according to claim 1 wherein the broncho- effector is selected from a group including: (a) chemical effectors;
(b) environmental effectors; (c) mechanical effectors; and (d) biological effectors.

7. A method according to claim 1 including the step of continuously monitoring the broncho-dynamic response over a period of time.

8. A method according to claim 1 wherein the sound signal is introduced into the airway continuously or intermittently.

9. A method according to claim 1 including the step of correcting the response to the broncho-effector for effects on the responsive sound signal which are attributable to changes in lung volume due to respiration.

10. A method according to claim 9 wherein changes in lung volume due to respiration affect at least one of a responsive sound signal magnitude and a propagation delay, and the correcting step includes at least one of:
(a) characterizing a changing volume-effect of respiration on the responsive sound signal magnitude and applying a magnitude correction factor to the responsive sound signal based on the magnitude characterization; and
(b) characterizing the changing volume-effect of respiration on the responsive sound signal propagation delay and applying a propagation delay correction factor to the responsive sound signal, based on the propagation delay characterization.

11. A method according to claim 9, including the step of determining an acoustic transmission of the subject, and further wherein:
(i) a given decrease in acoustic transmission in response to the broncho-effector is used to quantify a broncho-constrictive response in the subject; and
(ii) a given increase in acoustic transmission in response to the broncho-effector is used to quantify a broncho-dilating response in the subject,
wherein the correcting step includes:
(i) estimating a lung volume at regular intervals during monitoring of the responsive signals; and
(ii) depending on the lung volume estimate at each interval, applying a correction factor to at least one of the magnitude and the propagation delay of the acoustic transmission; wherein the correction factor is determined based on one or more relationships characterizing the changing lung volume effect of respiration on acoustic transmission magnitude and/or propagation delay.

12. A method according to claim 11 wherein the correction factor is different for different sound signal frequencies or bands of frequencies.

13. A method according to claim 9 wherein the correcting step is adaptive such that the correction factor varies according to changes in the broncho-dynamic response.

14. A method according to claim 9 wherein the correcting step is performed in real time or in an analysis step performed after the one or more responsive signals has been detected and stored on a storage medium.

15. A method according to claim 1 wherein the sound signal comprises frequencies in the audible range.

16. A method according to claim 1 wherein the sound signal is introduced in: (a) the mouth; (b) one or both nares; (c) endotracheal; or (d) using a face mask.

17. A method according to claim 1 including the step of providing oxygen-enriched gas mixture for delivery into the airway of the subject during the assessment.

18. Apparatus for assessing broncho-dynamic response in a subject, the apparatus including:
(a) an acoustic signal generator generating a sound signal having known characteristics;
(b) a sound introducer for introduction of sound into an airway of the subject;
(c) one or more sound transducers for detecting one or more responsive sound signal at one or more locations on a body of the subject;
(d) a dosimeter for delivering controlled dosages of broncho-effector to the subject; and
(e) a processor configured to receive the one or more responsive sound signals from the one or more sound transducers and calculate a response to the broncho-effector, based on changes in the one or more responsive sound signal characteristics.

19. Apparatus according to claim 18 wherein the processor is configured to receive the responsive sound signals and calculate the response to the broncho-effector in real-time.

20. Apparatus according to claim 18 including a reference transducer locatable on a neck of the subject, just below a glottis.

21. Apparatus according to claim 18 wherein the processor is configured to calculate acoustic transmission representing a respiratory response to the broncho-effector, and wherein: (i) a given decrease in acoustic transmission is used to quantify a broncho-constrictive response in the subject's airway; and (ii) a given increase in acoustic transmission is used to quantify a broncho-dilating response in the airway.

22. Apparatus according to claim 18 wherein the dosimeter is configured to expose the subject to controlled doses of a broncho-effector selected from a group including: (a) chemical effectors; (b) environmental effectors; (c) mechanical effectors; and (d) biological effectors.

23. Apparatus according to claim 18 wherein the controlled dosage is determined based, at least in part, on the response to a previously administered dosage.

24. Apparatus according to claim 18 wherein the processor is adapted to obtain dynamic measurements of the response, wherein the response is monitored over a period of time.

25. Apparatus according to claim 18 including means for determining changes in lung volume due to respiration.

26. Apparatus according to claim 18 wherein the processor is adapted to compensate a monitored response for the effect on the one or more responsive sound signal characteristics of changes in lung volume due to respiration.

27. Apparatus according to claim 25, wherein the processor is configured to calculate acoustic transmission representing the respiratory response to the broncho-effector, and wherein: (i) a given decrease in acoustic transmission is used to quantify a broncho-constrictive response in the airway; and (ii) a given increase in acoustic transmission is used to quantify a broncho-dilating response in the airway
and further wherein the processor is configured to (i) estimate lung volume at regular intervals during monitoring of the responsive sound signals; and (ii) apply a correction factor to the calculated acoustic transmission, the correction factor being based on a relationship characterizing the changing volume effect of respiration on the respiratory acoustic transmission.

28. Apparatus according to claim 27, wherein the processor applies a different correction factor for one or more sound signal frequencies or bands of frequencies.

29. Apparatus according to claim 27 wherein the processor varies the correction factor applied to the acoustic transmission according to changes in the broncho-dynamic response.

30. Apparatus according to claim 18 further including a muffler applicable to at least one of the acoustic signal generator and the sound introducing means to reduce sound emitted to the environment external to the subject.

31. Apparatus according to claim 18 wherein the sound introducing means is selected from a group including: (a) a mask; (b) a nasal cannula in communication with one or both nares; (c) an endotracheal tube; and (d) a mouthpiece.

32. Apparatus according to claim 18 wherein the processor is configured to calculate a value indicating a sensitivity to the administered broncho-effector.

33. Apparatus according to claim 18 including provision for introducing oxygen-enriched gas mixture into the airway of the subject.
